(19) United States
(12) Patent Application Publication
(10) Pub. No.: US 2025/0205064 A1
(43) Pub. Date: Jun. 26, 2025

(54) VASCULAR STENT

(71) Applicant: NEWTEC VASCULAR PRODUCTS LTD, Liverpool (GB)

(72) Inventor: Stephen Barker, Liverpool (GB)

(21) Appl. No.: 18/844,753

(22) PCT Filed: Mar. 8, 2023
(86) PCT No.: PCT/GB2023/050542

§ 371 Date: Sep. 6, 2024

(30) Foreign Application Priority Data
Mar. 8, 2022 (GB) 2203200.7

(51) Int. Cl. A61F 2/856; A61B 17/11; A61F 2/00

(52) U.S. Cl. CPC A61F 2/856; A61B 17/11; A61B 2017/1107; A61B 2017/1135; A61F 2002/0081; A61F 2210/0004; A61F 2210/0076; A61F 2230/0008; A61F 2230/0013

(57) ABSTRACT

A vascular stent for placement around an outside of a circulatory anastomosis is disclosed. The vascular stent comprises a laminated body having a biodegradable inner collagen layer and a perforated outer collagen layer arranged around the biodegradable inner collagen layer. Also disclosed are a method of using the vascular stent in an arteriovenous bypass grafting procedure and a kit comprising the vascular stent and instructions for using the vascular stent in an arteriovenous bypass grafting procedure.

### BACKGROUND

Neo-intimal hyperplasia represents an increase in the number of smooth muscle cells (SMC's) between the endothelium and the internal elastic lamina of a blood vessel.

When intimal hyperplasia occurs, de novo thickening of the intimal layer, or vessel wall may result, causing the vessel to become quickly and progressively stenosed, or even occluded (often in association with thrombosis). Proliferation of arterial SMC's commonly occurs when a blood vessel has its intima damaged, deformed, or disturbed during surgery. Surgical anastomoses, in particular associated with bypass grafts (in which a vein, or synthetic substitute is anastomosed to an artery), typically may result in SMC proliferation and consequently, stenosis.

A significant number of arterial bypass grafts fail, i.e. become occluded, in the first one to six months following surgery, but occlusion may still occur up to two years following. In these cases, it is SMC intimal hyperplasia that often is responsible for causing stenosis of the vessel lumen eventually resulting perhaps, in complete occlusion. SMC intimal hyperplasia occurs most commonly around the more distal (usually venous end) anastomosis and in the “native” vessel wall opposite the anastomosis. SMC intimal hyperplasia can occur at the proximal arterial anastomosis also and along parts of the graft itself.

A graft is commonly anastomosed to the native vessel in one of three ways: “end-to-end”, “end-to-side”, or “side-to-side”. Of these techniques, end-to-side and side-to-side are more common than end-to-end.

WO 2007/010295 A1 discloses an external vessel stent having an essentially non-porous outer layer and a biodegradable inner layer. The outer layer is relatively rigid and provides a structural framework, whereas the inner layer is relatively fast-degrading.

When the stent of WO 2007/010295 A1 is implanted around an anastomosis, the stent attracts serous fluid and new micro-vessels begin to grow primarily as a result of its internal collagen sponge construction. These micro-vessels, which are initially single cell wall thick endothelial cells, tend ultimately to produce large amounts of nitric oxide, which is known to be directly inhibitory on the build-up of SMCs on the inside of the anastomosis. Increasing the rate at which micro-vessels form will therefore reduce the build-up of SMCs, limiting SMC neointimal hyperplasia and hence, increase the success rate of vascular bypass grafts.

### SUMMARY OF THE INVENTION

According to a first aspect of the invention, there is provided a vascular stent for placement around an outside of a circulatory anastomosis; the vascular stent comprising a laminated body having a biodegradable inner collagen (sponge) layer and a perforated outer (dense) collagen (sheet) layer arranged around the biodegradable inner collagen layer.

When using the prior art stent of WO 2007/010295 A1, the outer collagen layer is so dense that it will take several months for it to be phagocytosed by macrophage cells circulating in the blood stream, whereas the inner collagen layer is relatively biodegradable and will be phagocytosed ideally in around one to four weeks. However, during the first few days (before the inner collagen layer has been phagocytosed), the surface area of the inner collagen layer presented to the early micro-vessels (and hence macrophage cells) is relatively small (i.e. only the ends of the inner collagen layer). This therefore restricts the production of nitric oxide during the first few days after the stent is implanted at surgery.

Using a perforated outer collagen layer in the present invention allows micro-blood vessel ingrowth from the outside of the device and increases the size of surface area of the unperforated inner collagen layer that is presented to the micro-vessels from the outset and which therefore can be immediately phagocytosed. This in turn encourages the growth of nitric oxide producing cells, thereby both increasing the overall production of nitric oxide and improving the distribution of nitric oxide along the length of the stent. The perforated outer layer therefore reduces the build-up of SMCs and increases the success rate of arterial bypass grafts compared to the stent of WO 2007/010295 A1.

A circulatory anastomosis is an anastomosis between two blood vessels (e.g. between two veins, two arteries, or between a vein and an artery, or between a vessel and a bypass graft which itself may be a natural or synthetic material). Any reference to an anastomosis herein should be understood to refer to a circulatory anastomosis.

Laminated means that the vascular stent is formed of bonded layers (i.e. the inner collagen and the outer collagen layer).

The vascular stent may be referred to herein simply as a stent, or it may be referred to as an external stent or external vascular stent, where the term “external” refers to the fact that the stent is for placement around the outside of (rather than within) a blood vessel.

The inner and/or outer collagen layers may be formed from a collagen material derived form a bovine, or porcine, or other natural or man-made source and are preferably predominantly Type I collagen. The outer collagen layer may be a dermal, pericardium or peritoneum collagen membrane, for example.

The outer collagen layer comprises a plurality of holes (i.e. through-holes or perforations). The holes may be substantially elliptical (e.g. circular), or they may have other regular or irregular shapes.

Preferably, each hole of the plurality of holes has a diameter (i.e. the distance between opposing sides the hole) of 0.1 mm to 5.0 mm, even more preferably 0.1 mm to 0.5 mm.

Using holes this size allows micro-vessels and hence, macrophages to reach the inner collagen layer whilst also ensuring that the outer collagen layer is not structurally weakened (the outer collagen layer acts as a scaffold element around the anastomosis) and preventing the inner collagen layer leaking through holes (at the time of manufacture).

Preferably, a distance (separation) between adjacent (i.e. nearest neighbour) holes is 1 mm to 10 mm. The holes may be positioned regularly (e.g. in a grid), or irregularly (e.g. with different spacing between different pairs of adjacent holes). A density of the holes may be between 1 hole per mm² and 0.01 holes per mm² (i.e. 1 hole per 100 mm²).

Using this separation distance (or density) ensures that the growth of micro-vessels (and therefore the production of nitric oxide) is distributed evenly along the stent length whilst also maintaining the structural integrity of the outer collagen layer.

The vascular stent is preferably elongate (i.e. long in relation to its width). It may be cut so as to provide longer and shorter lengths which in turn, can be placed suitably around varying forms of vascular anastomosis.

A profile of the vascular stent may be elliptical (e.g. oval or circular) or arcuate in a plane perpendicular to a longitudinal axis of the vascular stent (i.e. the vascular stent may have an elliptical or arcuate cross section through an axial extent of the vascular stent).

Having an elliptical (e.g. circular) profile (i.e. being formed as a tube) means that the stent must be implanted during a bypass procedure by threading it over one of the blood vessels before the two vessels are connected (e.g. during an end-to-end connection).

Having an arcuate profile means that the stent can instead be implanted after the vessels are connected, e.g. by opening the vascular stent (using a cut lengthwise along the stent) and placing it around the vessel, or by placing the stent on the vessel like a saddle.

The angle subtended by the arcuate portion may be less than 270°.

Preferably, the outer collagen layer has a thickness of 0.1 mm to 1.0 mm. This thickness allows the outer collagen layer to have enough strength to support the inner collagen layer and the anastomosis whilst still being easy to handle and implant. This thickness also helps to ensure that the outer layer has a suitable degradation profile.

Preferably, the outer collagen layer has a degradation profile such that it is retained for at least three months after implantation in a human or animal body and preferably, for between three to six months. That is, the characteristics of the outer collagen layer (e.g. the thickness and material) are chosen such that the structural integrity of the outer collagen layer will be maintained during at least the first three months following implantation of the stent in a human or animal body.

Preferably, the inner collagen layer has a degradation profile such that it is retained for a minimum of seven days and/or for a maximum of 28 days after implantation in a human or animal body. That is, the characteristics of the inner collagen layer (e.g.

the thickness and material) are chosen such that the structural integrity of the inner collagen layer will be maintained during the first seven days following implantation of the stent in a human or animal body but will be fully degraded within 28 days following implantation of the stent in a human or animal body.

A person skilled in the art will recognise that there are numerous materials and thicknesses that can achieve the required degradation profiles, and they will select the characteristics of the inner and outer collagen layers accordingly.

Preferably, the vascular stent has a length of 55 mm to 65 mm (e.g. about 60 mm). The vascular stent may be cut to a shorter length during surgery as required.

Optionally, the vascular stent may be formed of an elongate portion; and, a tubular portion protruding from a side of the elongate portion, such that, in use, the tubular portion is positionable around a graft vessel of an end-to-side anastomosis and the elongate portion is positionable around a native vessel of the end-to-side anastomosis. Such a vascular stent may have a Y-shape or T-shape. A more disc-like version may be constructed so as to sit covering a side-to-side anastomosis.

The tubular portion is preferably formed of the same inner and outer collagen layers as the elongate portion.

According to a second aspect of the invention, there is provided a method of using the vascular stent of the first aspect in an arteriovenous bypass grafting procedure. The procedure may optionally involve the use of a synthetic graft material.

According to a third aspect of the invention, there is provided a kit comprising: the vascular stent of the first aspect; and, instructions for using the vascular stent in an arteriovenous bypass grafting procedure.

### BRIEF DESCRIPTION OF THE DRAWINGS

Examples of the present invention will now be described in detail with reference to the accompanying drawings, in which:

FIG. 1a shows an external stent positioned at the site of an end-to-side anastomosis;

FIG. 1b shows an external stent positioned at the site of an end-to-end anastomosis;

FIG. 2a shows a cross sectional view of the external stent of FIG. 1a;

FIG. 2b shows a cross sectional view of the external stent of FIG. 1b;

FIG. 3a shows an outer layer of the external stent of FIG. 1a or 1b (not to scale);

FIG. 3b shows a cross-sectional view of the external stent of FIG. 1a or 1b, including a perforated outer layer.

FIG. 4a shows a photograph of a collagen layer (dermis) without perforations;

FIG. 4b shows a magnified view of the photograph of FIG. 4b:

FIG. 4c shows a photograph of an outer layer (dermis) of an external stent with perforations;

FIG. 4d shows a magnified view of the photograph of FIG. 4c; and,

FIGS. 5a-e show scale photographs of example perforations in an outer collagen layer (dermis).

### DETAILED DESCRIPTION

The present invention provides an improved external vascular stent for supporting a vascular join (anastomosis). Bypass surgery uses either sections of a patient's own healthy vessels (usually veins), or synthetic grafts, to reconnect the blood supply when blood flow is restricted (e.g. due to smooth muscle cell neo-intimal hyperplasia (SMCNIH), or atherosclerosis). As the re-joined vessels heal, there is a tendency for ‘scar’ tissue to overgrow (SMCNIH) on the inside of the vessel, causing it to constrict at the join, resulting in reduced blood flow, turbulence and high risk of clot formation (thrombosis). The external stent of the present invention device inhibits blood vessel SMCNIH tissue overgrowth and promotes optimal healing of the joined vessels.

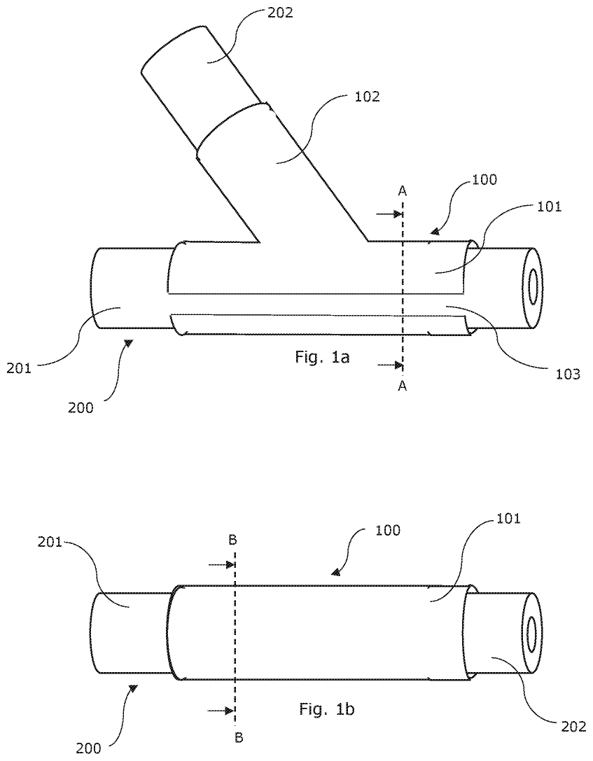

FIG. 1a illustrates a Y-shaped stent 100 according to the present invention. The stent 100 is placed around a blood vessel 200 formed of a graft vessel 202 connected to a native vessel 201 at an anastomosis site in an end-to-side configuration. The stent 100 has an elongation portion 101 positioned around the native vessel 101, and a tubular portion 102 protruding from the side of the elongate portion 101 and positioned around the graft vessel 202. While the illustrated stent 100 has the tubular portion 102 protruding at an acute angle relative to the elongate portion 101, it could alternatively protrude at a different angle such as a right angle (thereby giving the stent a T-shape).

The illustrated stent 100 also features a slit 103 in the elongate portion 101, which results in the elongate portion having an arcuate cross section when viewed in a plane perpendicular to the longitudinal axis of the elongate portion 101. Although the illustrated stent 100 surrounds the majority of the outer surface of the native vessel 201, the size of the slit 103 (and the resulting angle subtended by the arcuate cross section) may be varied. For example, the slit could be much larger so that the elongate portion 101 surrounds only a top half of the native vessel 201, i.e. such that the elongate portion 101 “saddles” the native vessel 101. The arcuate configuration of the elongate portion 101 allows the stent 100 to be placed around the native vessel 201 without needing to cut through the native vessel 101.

The tubular portion 102 is preferably elliptical (e.g. circular) in cross section and the graft vessel 202 is preferably threaded through the tubular portion 102 during the surgical procedure, although the tubular portion 102 could alternatively have an arcuate cross section similar to the elongate portion 101.

FIG. 1b illustrates an alternative tubular stent 100 according to the present invention. In FIG. 1b, the stent is positioned around a blood vessel 200 formed of a graft vessel 202 connected to a native vessel 201 at an anastomosis site in an end-to-end configuration. In this example, the stent 100 comprises only an elongate portion 101 and no tubular portion. As the stent 100 can be threaded over the native vessel 201 or graft vessel 202 during the surgical procedure, there is no need for a slit in the stent 100 shown in FIG. 1b. However, arrangements with a slit (pre-cut at device manufacture, or cut at the time of use during surgery) are also envisaged.

In both FIGS. 1a and 1b, the elongate portion 101 preferably has a length of 55 mm to 65 mm (e.g. 60 mm). In generally, once implanted there will ideally be an overlap of at least 2 cm in any direction away from the line of anastomosis, including over any synthetic graft used in surgery.

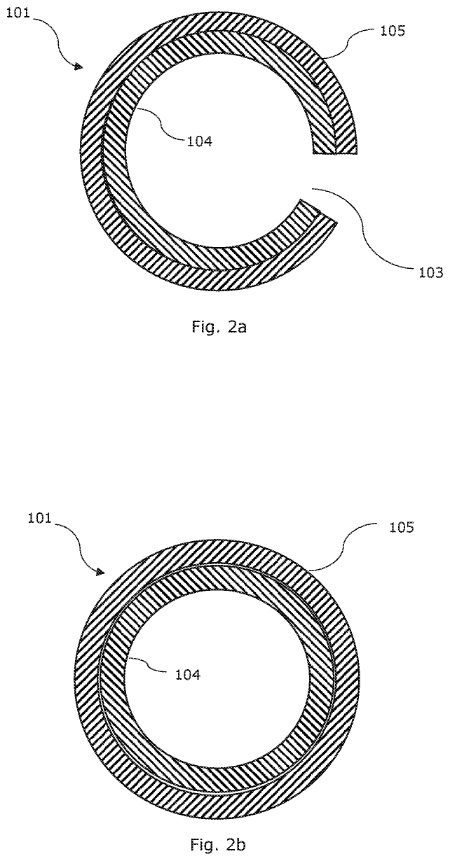

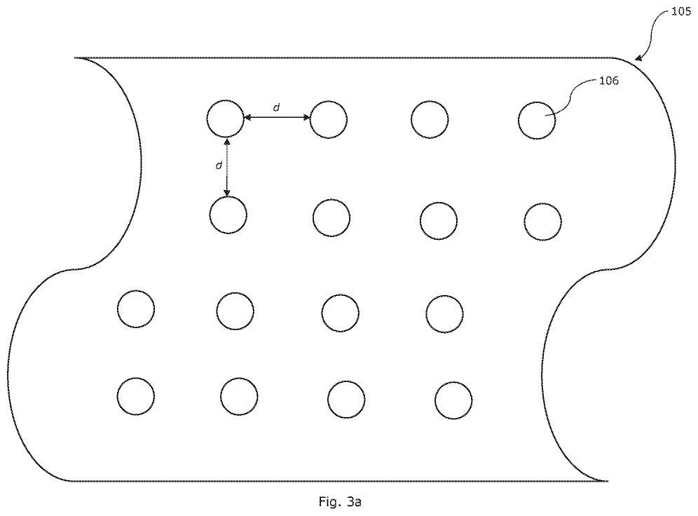

FIGS. 2a and 2b show cross sectional views of the stents 100 shown in FIGS. 1a and 1b respectively. FIG. 2a is a cross section taken through the section line A-A, and FIG. 2b is a cross section taken through the section line B-B.

As shown in both FIGS. 2a and 2b, the elongate portion 101 has a laminated structure and is formed of an inner collagen layer 104 bonded to a surrounding outer collagen layer 105. Although not illustrated, the tubular portion 102 (if present) will generally have the same laminated structure as the elongation portion 101.

The outer collagen layer 105 is relatively rigid and acts as a scaffold to support the anastomosis after surgery, and also acts as a substrate onto which the inner collagen layer 104 can be applied.

The outer collagen layer 105 is preferably formed from a dermal collagen membrane derived from a bovine (certified BSE free), or porcine source which will preferably have undergone a viral inactivation treatment. The thickness of the outer collagen layer is preferably 0.1 mm to 1.0 mm.

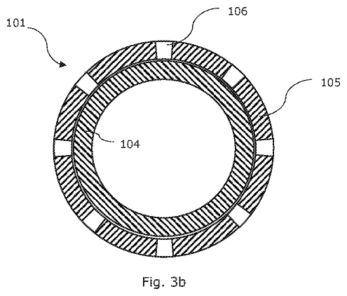

In WO 2007/010295 A1, the outer layer was essentially non-porous. In contrast, the stent 100 of the present invention features a perforated outer collagen layer 105. As shown in FIG. 3b, the outer collagen layer 105 only is perforated and the inner collagen layer 104 is unperforated. Using a perforated outer collagen layer 105 allows micro-blood vessel ingrowth from the outside of the device and increases the size of surface area of the inner collagen layer that is presented to the micro-vessels from the outset and which therefore can be immediately phagocytosed. Correspondingly, the inner collagen layer 104 is left unperforated in order to maximise the amount of sponge material available for bio-degradation. The combination of perforated outer layer 105 and unperforated inner layer 104 ensures that the inner layer 104 biodegrades at a faster rate than the outer layer 105, resulting in an increased success rate for arterial bypass grafts.

As shown in FIG. 3a (which is not to scale), the perforated outer collagen layer 105 has a plurality of holes 106 with a preferable diameter of 0.1 mm to 5.0 mm. The spacing between adjacent/nearest neighbour holes (labelled d in FIG. 3) is preferably 1 mm to 10 mm. The holes 106 may be formed using any suitable process.

As shown in FIG. 3b, in an embodiment the holes 106 may extend all the way through the perforated outer layer 105, but not into the unperforated inner layer 104. The holes 106 may be arranged symmetrically and equidistantly around the circumference of the perforated outer layer as shown, but other arrangements are also envisaged.

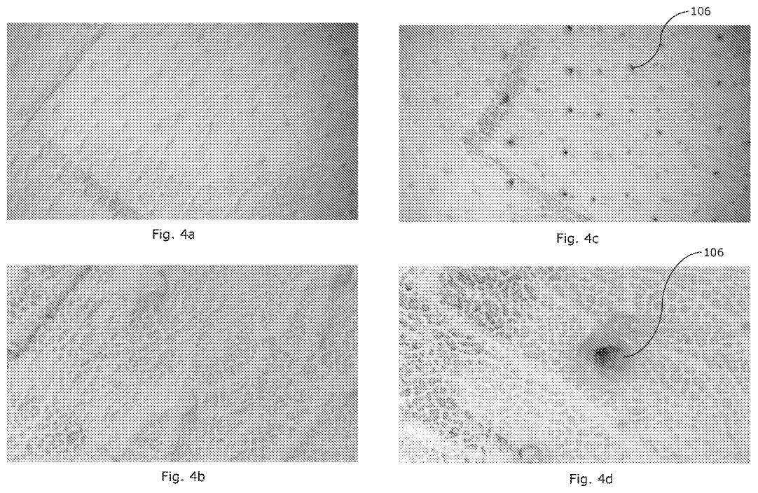

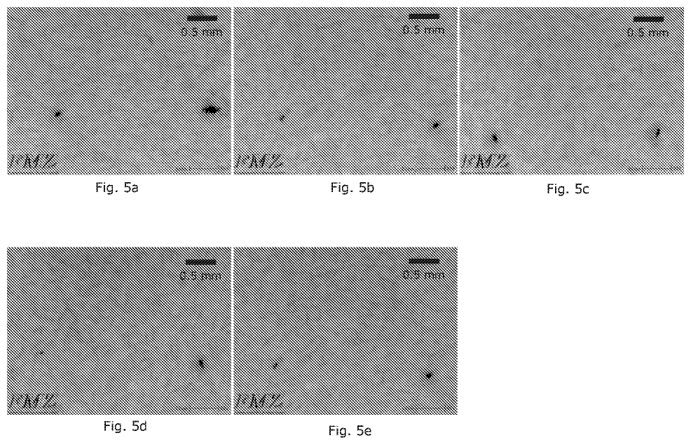

FIGS. 4a and 4b show an exemplary collagen substrate (dermis) without holes, and FIGS. 4c and 4d show an exemplary collagen substrate (dermis) provided with holes 106. Further examples of collagen layers with holes that are suitable for use as outer collagen layers 105 in the present invention are shown in FIGS. 5a-5e.

The outer collagen layer 105 has a degradation profile such that the structure after implantation should be retained for a minimum of three months.

The inner collagen layer 104 is biodegradable (i.e. degrades at a faster rate than the outer collagen layer 105 after implantation) and is provided within the outer collagen layer 105 such that an inner surface of the outer collagen layer is in contact with an outer surface of the inner collagen layer. The inner collagen layer 104 has a degradation profile such that the structure after implantation should ideally be mostly retained for seven days and for a maximum of 28 days.

In addition, the inner collagen layer 104 has a composition that permits good adhesion to the outer collagen layer 105 during processing, and the outer collagen layer 105 has a structure that permits good adhesion of the inner collagen layer 104 during processing.

According to an exemplary embodiment, the collagen material used for the inner collagen layer may be prepared as a suspension in sterile water. It may then optionally be filtered prior to injection into the centre of a mould bounded by the outer collagen layer 105. The resulting stent 100 will ideally have a confluent coating of collagen material (the inner collagen layer 104) throughout the outer collagen layers 105 of the elongate portion 101 and tubular portion 102, with a consistent/uniform thickness and no gaps in the coating.

As with the outer collagen layer 105, the inner collagen layer 104 is preferably formed from a collagen material from a bovine (certified BSE free) or porcine source which will preferably have undergone a viral inactivation treatment.

The stent 100 of the present invention is a single-use device and is preferably distributed in sealed, sterile packing as part of a kit comprising the stent 100 and instructions for use (IFUs).

1-13. (canceled)

14. A vascular stent for placement around an outside of a circulatory anastomosis, the vascular stent comprising a laminated body having a biodegradable inner collagen layer and a perforated outer collagen layer arranged around the biodegradable inner collagen layer, wherein h perforated outer collagen layer comprises a plurality of holes and each hole of the plurality of holes has a diameter of 0.1 mm to 5.0 mm.

15. The vascular stent of claim 14, wherein each hole of the plurality of holes has a diameter of 0.1 mm to 0.5 mm.

16. The vascular stent of claim 15, wherein a distance between adjacent holes is 1 mm to 10 mm.

17. The vascular stent of claim 14, wherein a profile of the vascular stent is elliptical or arcuate in a plane perpendicular to a longitudinal axis of the vascular stent.

18. The vascular stent of claim 14, wherein the perforated outer collagen layer has a thickness of 0.1 mm to 1.0 mm.

19. The vascular stent of claim 14, wherein the perforated outer collagen layer has a degradation profile such that it is retained for at least three months after implantation in a human or animal body.

20. The vascular stent of claim 14, wherein the biodegradable inner collagen layer has a degradation profile such that it is retained for a minimum of seven days after implantation in a human or animal body.

21. The vascular stent of claim 14, wherein the biodegradable inner collagen layer has a degradation profile such that it is retained for a maximum of 28 days after implantation in a human or animal body.

22. The vascular stent of claim 14, wherein the vascular stent has a length of 55 mm to 65 mm.

23. The vascular stent of claim 14, wherein the vascular stent is formed of:
an elongate portion; and
a tubular portion protruding from a side of the elongate portion,
such that, in use, the tubular portion is positionable around a graft vessel of an end-to-side anastomosis and the elongate portion is positionable around a native vessel of the end-to-side anastomosis.

24. A kit comprising:
the vascular stent of claim 14; and
instructions for using the vascular stent in an arteriovenous bypass grafting procedure.

25. An arteriovenous bypass grafting procedure performed on a patient, comprising the placement of a vascular stent around an outside of a circulatory anastomosis,
wherein the vascular stent comprises a laminated body having a biodegradable inner collagen layer, and a perforated outer collagen layer arranged around the biodegradable inner collagen layer, and
wherein the perforated outer collagen layer comprises a plurality of holes and each hole of the plurality of holes has a diameter of 0.1 mm to 5.0 mm.

26. The procedure of claim 25, wherein each hole of the plurality of holes has a diameter of 0.1 mm to 0.5 mm.

27. The procedure of claim 25, wherein a distance between adjacent holes is 1 mm to 10 mm.

28. The procedure of claim 25, wherein a profile of the vascular stent is elliptical or arcuate in a plane perpendicular to a longitudinal axis of the vascular stent.

29. The procedure of claim 25, wherein the perforated outer collagen layer has a thickness of 0.1 mm to 1.0 mm.

30. The procedure of claim 25, wherein the perforated outer collagen layer has a degradation profile such that it is retained for at least three months after implantation in a human or animal body.

31. The procedure of claim 25, wherein the biodegradable inner collagen layer has a degradation profile such that it is retained for a minimum of seven days after implantation in a human or animal body.

32. The procedure of claim 25, wherein the biodegradable inner collagen layer has a degradation profile such that it is retained for a maximum of 28 days after implantation in a human or animal body.
